(19) United States
(12) Patent Application Publication
(10) Pub. No.: US 2007/0009533 A1
(43) Pub. Date: Jan. 11, 2007

(54) Treatment of cancer patients exhibiting activation of the P-glycoprotein efflux pump mechanism

(76) Inventors: Branimir Sikic, Stanford, CA (US); Daniel Hoth, San Francisco, CA (US); David Socks, Carlsbad, CA (US); Scott Glenn, La Jolla, CA (US); John Marcelletti, San Diego, CA (US); Michael J. Walsh, San Diego, CA (US); Pratik S. Multani, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/417,984

(22) Filed: May 3, 2006

Related U.S. Application Data
(60) Provisional application No. 60/696,756, filed on Jul. 6, 2005.
(60) Provisional application No. 60/726,827, filed on Oct. 14, 2005.

(51) Int. Cl. A61K 39/395; A61K 38/13; A61K 31/553; A61K 31/551; A61K 31/455; A61K 31/4709; A61K 31/704; A61K 31/277; A61K 31/138

(52) U.S. Cl. 424/155.1; 514/220; 514/11; 514/26; 514/263.32; 514/317; 514/469; 514/253.04; 514/389; 514/291; 514/225.8; 514/314; 514/355; 514/211.07; 514/521; 514/651

(57) ABSTRACT

The present invention relates to a method of determining P-glycoprotein expression and/or function for a patient with solid tumors, leukemias, and other malignancies. The invention also relates to using a P-glycoprotein expression and/or function diagnostic in conjunction with methods for treating solid tumors, leukemias, and other malignancies with a chemotherapeutic agent in combination with zosuquidar. The methods are particularly effective in treating acute myelogenous leukemia, metastatic breast cancer, and other cancers expressing P-glycoprotein, wherein the P-glycoprotein expression and/or function is used to select a treatment option for the patient.

### RELATED APPLICATION

This application claims priority under 35 U.S.C. §119(e) to U.S. Provisional Application No. 60/696,913 filed Jul. 6, 2005, and to U.S. Provisional Application No. 60/726,827 filed Oct. 14, 2005, which are incorporated by reference herein in their entirety, and which are hereby made a part of this specification.

### FIELD OF THE INVENTION

The present invention relates to a method of determining P-glycoprotein expression and/or function for a patient with solid tumors, leukemias, and other malignancies. The invention also relates to using a P-glycoprotein expression and/or function diagnostic in conjunction with methods for treating solid tumors, leukemias, and other malignancies with a chemotherapeutic agent in combination with zosuquidar. The methods are particularly effective in treating acute myelogenous leukemia, metastatic breast cancer, and other cancers expressing P-glycoprotein, wherein the P-glycoprotein expression and/or function is used to select a treatment option for the patient.

### BACKGROUND OF THE INVENTION

The field of oncology is in the midst of a major evolution. In the past, the treatment of cancer has been dominated by empiric, “one-size-fits-all” treatments based on types and stages of tumors. Toxic chemotherapy drugs have dominated the treatment landscape despite a very low cure rate, particularly against the most common cancers and those with known metastatic disease.

Now, treatments in development are targeted against specific proteins. Such targeting is based on a more robust knowledge of cancer mechanisms, which often crosses over many tumor types. These treatments are designed to work in defined subsets of patients, typically based on expression and function of the target protein rather than the type of tumor, and often in combination with standard chemotherapies. Advances in the molecular analysis of cancers will enable the identification of such susbsets of patients and the coupling of targeted therapeutics to novel diagnostic approaches.

The future of oncology lies in defining the disease in molecular terms (i.e., genetics, genomics, proteomics) and tailoring therapies according to individual tumor and normal cell properties. This new paradigm will predetermine likely responders, assess responses earlier, and adjust treatment based on continued molecular analyses of tumors.

Drug resistance is one of the most difficult problems that must be overcome in order to achieve successful treatment of human tumors with chemotherapy. Clinically, drug resistance, a characteristic of intrinsically resistant tumors (for example, colon, renal, and pancreas), may be evident at the onset of therapy. Alternatively, acquired drug resistance results when tumors initially respond to therapy but become refractory to subsequent treatments. Once a tumor has acquired resistance to a specific chemotherapeutic agent, it is common to observe collateral resistance to other structurally similar agents. The cellular mechanisms of drug resistance include apoptosis, drug uptake, DNA repair, altered drug targets, drug sequestration, detoxification, and efflux pumps (see, e.g., Dalton W. S. Semin. Oncol. 20:60, 1993).

Multidrug resistance (MDR), the ability of cancer cells to become resistant to the agent(s) actively used for therapy as well as other drugs that are structurally and functionally unrelated, is a particularly insidious form of drug resistance. This form of drug resistance is discussed in greater detail in Kuzmich et al., “Detoxification Mechanisms and Tumor Cell Resistance to Anticancer Drugs,” particularly section VII “The Multidrug-Resistant Phenotype (MDR),” Medical Research Reviews, Vol. 11, No. 2, 185-217, particularly 208-213 (1991); and in Georges et al., “Multidrug Resistance and Chemosensitization: Therapeutic Implications for Cancer Chemotherapy,” Advances in Pharmacology, Vol. 21, 185-220 (1990).

Although MDR may be caused by a variety of factors, one of the most prevalent forms of MDR is the type associated with overexpression of P-glycoprotein (P-gp). P-gp is a member of a superfamily of membrane proteins, termed adenosine triphosphate (ATP)-binding cassette (ABC) proteins, which behave as ATP-dependent transporters and/or ion channels for a wide variety of hydrophobic substrates. P-gp is a multiple transmembrane-spanning glycoprotein. Transfection experiments with the P-gp gene (MDR1, or ABCB1) have conferred MDR to drug-sensitive tumor cells by providing an energy-dependent efflux pump that lowers the intracellular concentration of the cytotoxic agent, thereby allowing survival of the cell.

P-gp is expressed in normal biliary canaliculi of the liver, the adrenal cortex and proximal tubules of the kidney, and intestinal epithelia including the columnar cells of the large and small intestines; capillary endothelial cells of brain, testis, and placenta; and in the hematopoietic stem cells of bone marrow. It possesses excretory, protective, and barrier functions. P-gp is constitutively expressed or selected in many human cancers, and confers resistance to therapeutic agents including anthracyclines (e.g., doxorubicin, daunorubicin, epirubicin, idarubicin, mitoxantrone), vincas (e.g., vincristine, vinblastine, vinorelbine, vindesine), Topoisomerase-II inhibitors (e.g., etoposide, teniposide), taxanes (e.g., paclitaxel, docetaxel), and others (e.g., Gleevec, Mylotarg, dactinomycin, mithramycin).

The relative promiscuity of drug transport by P-gp and other MDR-associated transporters inspired a wide search for compounds that would not be cytotoxic themselves but would inhibit MDR transport. The initial demonstration of verapamil as a P-gp inhibitor was followed by many additional compounds reported to inhibit drug transport and thus sensitize MDR cells to chemotherapeutic drugs. Variously called chemosensitizers, MDR reversal agents, modulators, or converters, these ‘first generation’ MDR drugs included compounds of diverse structure and function such as calcium channel blockers (e.g., verapamil), immunosuppressants (e.g., cyclosporin A), antibiotics (e.g., erythromycin), antimalarials (e.g., quinine), and others (e.g., biricodar, tariquidar, valspodar).

First generation MDR drugs were not specifically developed for inhibiting MDR. They often had other pharmacological activities, as well as a relatively low affinity for MDR transporters and thus were limited in application. For example, P-gp has a low affinity for verapamil, thus requiring cardiotoxic levels for full modulator activity. In spite of the fact that only low serum levels could be obtained in a Phase II trial, 5 of 22 patients responded to a combination of verapamil and VAD (vincristine, doxorubicin, and dexamethasone). Four of the responders had elevated P-gp expression and function. Thus, verapamil has demonstrated some clinical utility in overcoming drug resistance. Cyclosporin A alters the pharmacokinetics of coadministered cytotoxic agents, resulting in significantly increased exposure to the oncolytic, thus confounding the interpretation of clinical trials.

Further characterization of the P-gp pharmacophore led to the identification of ‘second generation’ modulators based on the first generation but specifically selected or designed to reduce the side effects of the latter by eliminating their non-MDR pharmacological actions. Compounds such as the R-enantiomers of verapamil (R-verapamil) and dexniguldipine did not fare any better as MDR drugs in clinical studies, most likely because their affinity towards P-gp still fell short of producing significant inhibition of MDR in vivo at tolerable doses.

A more promising second generation modulator with a higher affinity towards P-gp was valspodar, a non-immunosuppressive cyclosporin D derivative. While early trials were encouraging, further work revealed significant pharmacokinetic interactions with several anticancer drugs. Although discontinued by Novartis, valspodar was studied in a Phase III study in elderly patients with acute myelogenous leukemia. Enrollment in the valspodar arm was halted due to excessive early mortality, most likely due to the PK interactions. Although the number of patients was limited, patients in the control arm whose pretreatment cells exhibited valspodar-modulated dye efflux in vitro (n=22) had worse outcomes than those without efflux (n=11) (complete remission, nonresponse, and death rates of 41%, 41%, and 18%, compared with 91%, 9%, and 0%; P=0.03), but with valspodar outcomes were nearly identical (Baer 2002). Moreover, for patients with valspodar-modulated efflux, median disease-free survival was 5 months in the control arm and 14 months with valspodar (P=0.07).

A second generation MDR modulator with activity against both P-gp and MRP1 (another ABC transporter associated with multidrug resistance) was biricodar. Vertex studied the agent in multiple Phase II studies of soft tissue sarcomas, ovarian cancer, small cell lung cancer, and others. However, biricodar and valspodar are both substrates for the P450 isoenzyme 3A4. Competition between cytotoxic agents and the P-gp inhibitors for cytochrome P450 3A4 resulted in unpredictable PK interactions and resulted in increased serum concentrations of cytoxics and, therefore, greater toxicity to the patient. A common response of clinical researchers has been to reduce the dose of the cytotoxic agents. However, the PK interactions are unpredictable and cannot be determined in advance. As a result, cytotoxic serum levels were either too high resulting in excessive toxicity or too low resulting in decreased efficacy. In addition to inhibiting P-gp, many of the second generation modulators function as substrates for other transporters, particularly the ABC family, inhibition of which could lessen the ability of normal, healthy cells to protect themselves from the cytotoxic agents.

### SUMMARY OF THE INVENTION

In spite of the methodological possibilities for detecting expression of MDR1, the use of P-gp expression and functional tests to predict response to therapy has not been very successful. Specifically, while there have been indications of clinical utility for such a test, the results have not been sufficiently strong to warrant routine testing. What has been lacking is a statistical algorithm relating response potential to chemotherapy on a patient-specific basis with P-gp function to the modulatory effects of zosuquidar, the immunophenotype, and cytogenetics of the cancer cells, and clinical data such as patient age.

Zosuquidar selectively inhibits MDR1 activity, allowing for the accumulation of sufficient chemotherapeutic agent to allow for effective therapy and tumor cell killing. MDR1 may be present in the tumor cells at the time of diagnosis or it may be acquired during treatment or remission of the cancer. It is desirable to identify patients who would benefit most from zosuquidar therapy to target that patient population, i.e., MDR1-positive. A highly sensitive and reproducible diagnostic test for the functional expression of MDR1 is required to accomplish this goal.

Testing methodology is provided that improves the efficacy rates for any P-gp substrate in a patient population with demonstrated pump activation, resulting in enhanced cure rates, cancer free survival rates, and overall survival rates for oncolytic drugs.

Accordingly, in a first aspect, a method of treating a cancer is provided, the method comprising the steps of determining P-glycoprotein expression or P-glycoprotein function in the cancer cells; selecting a treatment for the patient, based on the P-glycoprotein expression; and administering the treatment, whereby the cancer is treated.

In an embodiment of the first aspect, P-glycoprotein expression is determined by an antibody assay, and wherein the step of selecting the treatment and administering the treatment comprises administering a P-glycoprotein efflux pump inhibitor in combination with a chemotherapeutic agent that is a substrate for P-glycoprotein efflux when positive P-glycoprotein expression is observed in at least about 10% of the cells tested in the antibody assay.

In an embodiment of the first aspect, positive P-glycoprotein expression is observed in from about 10% to about 25% of the cells tested in the antibody assay.

In an embodiment of the first aspect, P-glycoprotein expression is determined by an antibody assay, and wherein the step of selecting the treatment and administering the treatment comprises administering a chemotherapeutic agent that is a substrate for P-glycoprotein efflux in the absence of a P-glycoprotein efflux pump inhibitor when positive P-glycoprotein expression is observed for less than about 10% of the cells tested in the antibody assay.

In an embodiment of the first aspect, P-glycoprotein function is determined by a P-glycoprotein dye accumulation assay, and wherein the step of selecting the treatment and administering the treatment comprises administering a P-glycoprotein efflux pump inhibitor in combination with a chemotherapeutic agent that is a substrate for P-glycoprotein efflux when, in the P-glycoprotein dye accumulation assay, a ratio of dye taken up by cells in the presence of the P-glycoprotein efflux pump inhibitor to dye taken up by cells cultured in the absence of the P-glycoprotein efflux pump inhibitor is at least about 1:1.2, e.g., from about 1:1.2 to about 1:1.5.

In an embodiment of the first aspect, P-glycoprotein function is determined by a P-glycoprotein dye accumulation assay, and wherein the step of selecting the treatment and administering the treatment comprises administering a chemotherapeutic agent that is a substrate for P-glycoprotein efflux in the absence of a P-glycoprotein efflux pump inhibitor when, in the P-glycoprotein dye accumulation assay, a ratio of dye taken up by cells in the presence of the P-glycoprotein efflux pump inhibitor to dye taken up by cells cultured in the absence of the P-glycoprotein efflux pump inhibitor is less than about 1:1.2.

In an embodiment of the first aspect, P-glycoprotein function is determined by a P-glycoprotein dye efflux assay, and wherein the step of selecting the treatment and administering the treatment comprises administering a P-glycoprotein efflux pump inhibitor in combination with a chemotherapeutic agent that is a substrate for P-glycoprotein efflux when, in the P-glycoprotein dye efflux assay, an amount of dye eliminated from the cells in the presence of the P-glycoprotein efflux pump inhibitor divided by an amount of dye eliminated in the absence of P-glycoprotein efflux pump inhibitor is at least about 1:1.2, e.g., from about 1:1.2 to about 1:1.5.

In an embodiment of the first aspect, P-glycoprotein function is determined by a P-glycoprotein dye efflux assay, and wherein the step of selecting the treatment and administering the treatment comprises administering a P-glycoprotein efflux pump inhibitor in combination with a chemotherapeutic agent that is a substrate for P-glycoprotein efflux when, in the P-glycoprotein dye efflux assay, an amount of dye eliminated from the cells in an absence of the P-glycoprotein efflux pump inhibitor is at least about 30% higher than that contained in baseline control cells, and wherein cells incubated in a presence of the P-glycoprotein efflux pump inhibitor have dye levels at least about 30% higher than cells cultured in an absence of the P-glycoprotein efflux pump inhibitor, e.g., from about 30% to about 50% higher than cells cultured in an absence of the P-glycoprotein efflux pump inhibitor.

In an embodiment of the first aspect, P-glycoprotein function is determined by a P-glycoprotein dye efflux assay, and wherein the step of selecting the treatment and administering the treatment comprises administering a chemotherapeutic agent that is a substrate for P-glycoprotein efflux in the absence of a P-glycoprotein efflux pump inhibitor when, in the P-glycoprotein dye efflux assay, an amount of dye eliminated from the cells in the presence of the P-glycoprotein efflux pump inhibitor divided by an amount of dye eliminated in the absence of P-glycoprotein efflux pump inhibitor is less than about 1:1.2.

In an embodiment of the first aspect, P-glycoprotein function is determined by a P-glycoprotein dye efflux assay, and wherein the step of selecting the treatment and administering the treatment comprises administering a chemotherapeutic agent that is a substrate for P-glycoprotein efflux in the absence of a P-glycoprotein efflux pump inhibitor when, in the P-glycoprotein dye efflux assay, an amount of dye eliminated from the cells in an absence of the P-glycoprotein efflux pump inhibitor is less than about 30% higher than that contained in baseline control cells, and wherein cells incubated in a presence of the P-glycoprotein efflux pump inhibitor have dye levels less than about 30% higher than cells cultured in an absence of the P-glycoprotein efflux pump inhibitor.

In an embodiment of the first aspect, the P-glycoprotein efflux pump inhibitor is selected from the group consisting of zosuquidar, Tariquidar, and Tesmilifene.

In an embodiment of the first aspect, the cancer is acute myelogenous leukemia.

In an embodiment of the first aspect, the cancer is a carcinoma (e.g., ovarian cancer or breast cancer), a sarcoma, or a hematologic malignancy (e.g., acute lymphoblastic leukemia, chronic myeloid leukemia, plasma cell dyscrasias, lymphoma, or myelodysplasia).

In an embodiment of the first aspect, the chemotherapeutic agent is an anthracycline (e.g., doxorubicin, daunorubicin, epirubicin, idarubicin, or mitoxantrone).

In an embodiment of the first aspect, the chemotherapeutic agent is a Topoisomerase-II inhibitor (e.g., etoposide or teniposide).

In an embodiment of the first aspect, the chemotherapeutic agent is a vinca (e.g., vincristine, vinblastine, vinorelbine, or vindesine).

In an embodiment of the first aspect, the chemotherapeutic agent is a taxane (paclitaxel or docetaxel).

In an embodiment of the first aspect, the chemotherapeutic agent is selected from the group consisting of gleevec, dactinomycin, bisantrene, mitoxantrone, actinomyocin D, mithomycin C, mitramycin, methotrexate, adriamycin, mitomycin, and mithramycin, anthracene, and epipodophyllo-toxin.

In a second aspect, a pharmaceutical kit is provided, the kit comprising at least one dose of a P-glycoprotein efflux pump inhibitor selected from the group consisting of zosuquidar, Tariquidar, and Tesmilifene; directions for conducting a diagnostic for determining P-glycoprotein expression or P-glycoprotein function associated with the cancer; and directions for administering the P-glycoprotein efflux pump inhibitor and a chemotherapeutic agent that is a substrate for P-glycoprotein efflux to the patient to treat the cancer when P-glycoprotein expression or P-glycoprotein function is positive.

In a third aspect, a method of treating a condition in a patient by administering a therapeutic agent that is a substrate for P-glycoprotein efflux is provided, the method comprising the steps of determining P-glycoprotein expression or P-glycoprotein function; and administering the therapeutic agent in combination with a P-glycoprotein efflux pump inhibitor when P-gp expression is positive.

In an embodiment of the third aspect, P-glycoprotein expression is determined by an antibody assay, and wherein positive P-glycoprotein expression is observed in at least about 10% of the cells tested in the antibody assay.

In an embodiment of the third aspect, P-glycoprotein expression is determined by an antibody assay, and wherein positive P-glycoprotein expression is observed in from about 10% to about 25% of the cells tested in the antibody assay.

In an embodiment of the third aspect, P-glycoprotein function is determined by a P-glycoprotein dye accumulation assay, and wherein a ratio of dye taken up by cells in the presence of the P-glycoprotein efflux pump inhibitor to dye taken up by cells cultured in the absence of the P-glycoprotein efflux pump inhibitor is at least about 1:1.2, e.g., from about 1:1.2 to about 1:1.5.

In an embodiment of the third aspect, P-glycoprotein function is determined by a P-glycoprotein dye efflux assay, and wherein an amount of dye eliminated from the cells in the presence of the P-glycoprotein efflux pump inhibitor divided by an amount of dye eliminated in the absence of P-glycoprotein efflux pump inhibitor is at least about 1:1.2, e.g., from about 1:1.2 to about 1:1.5.

In an embodiment of the third aspect, P-glycoprotein function is determined by a P-glycoprotein dye efflux assay, and wherein an amount of dye eliminated from the cells in an absence of the P-glycoprotein efflux pump inhibitor is at least about 30% higher than that contained in baseline control cells, and wherein cells incubated in a presence of the P-glycoprotein efflux pump inhibitor have dye levels at least about 30% higher than cells cultured in an absence of the P-glycoprotein efflux pump inhibitor.

In an embodiment of the third aspect, the P-glycoprotein efflux pump inhibitor is selected from the group consisting of zosuquidar, Tariquidar, and Tesmilifene.

In an embodiment of the third aspect, the therapeutic agent comprises an immunosuppressant (e.g., cyclosporine, cyclosporine A, or tacrolimus).

In an embodiment of the third aspect, the therapeutic agent comprises a steroid (e.g., dexamethasone, hydrocortisone, corticosterone, triamcinolone, aldosterone, or methylprednisolone).

In an embodiment of the third aspect, the therapeutic agent comprises an antiepileptic (e.g. phenytoin).

In an embodiment of the third aspect, the therapeutic agent comprises an antidepressant (e.g., citalopram, thioperidone, trazodone, trimipramine, amitriptyline, or phenothiazines).

In an embodiment of the third aspect, the therapeutic agent comprises an antipsychotic (e.g., fluphenazine, haloperidol, thioridazine, and trimipramine).

In an embodiment of the third aspect, the therapeutic agent comprises a protease inhibitor (e.g., amprenavir, indinavir, lopinavir, nelfinavir, ritonavir, or saquinavir).

In an embodiment of the third aspect, the therapeutic agent comprises a calcium blocker (e.g., bepridil, diltiazem, flunarizine, lomerizine, secoverine, tamolarizine, verapamil, nicardipine, prenylamine, or fendiline).

In an embodiment of the third aspect, the therapeutic agent comprises a cardiac drug (e.g., digoxin, diltiazem, verapamil, and talinolol).

In a fourth aspect, a pharmaceutical kit is provided, the kit comprising at least one dose of a P-glycoprotein efflux pump inhibitor selected from the group consisting of zosuquidar, Tariquidar, and Tesmilifene; directions for conducting a diagnostic for determining a value for P-gp expression associated with a condition; and directions for administering the P-glycoprotein efflux pump inhibitor and a therapeutic agent that is a substrate for P-glycoprotein efflux to the patient to treat the condition when P-glycoprotein expression or P-glycoprotein function is positive.

In a fifth aspect, a method of treating a cancer in a patient is provided, the method comprising the steps of conducting a P-glycoprotein efflux assay on the cancer cells in the presence of a P-glycoprotein efflux inhibitor, whereby a first value for P-glycoprotein function is obtained; conducting a P-glycoprotein efflux assay on the cancer cells in the absence of a P-glycoprotein efflux inhibitor, whereby a second value for P-glycoprotein function is obtained; comparing the first value and the second value for P-glycoprotein function, wherein the patient exhibits inhibitable P-glycoprotein efflux when the first value is greater than the second value; and administering to the patient exhibiting inhibitable P-glycoprotein efflux the P-glycoprotein efflux inhibitor and a chemotherapeutic agent that is a substrate for P-gp efflux.

In an embodiment of the fifth aspect, the patient exhibits inhibitable P-glycoprotein efflux when a ratio of the second value to the first value is greater than or equal to about 1:1.2, e.g., from about 1:1.2 to about 1:1.5.

In an embodiment of the fifth aspect, the patient exhibits inhibitable P-glycoprotein efflux when the first value is at least about 30% higher than the second value, e.g., from about 30% to about 50% higher than the second value.

In an embodiment of the fifth aspect, the P-glycoprotein efflux pump inhibitor selected from the group consisting of zosuquidar, Tariquidar, and Tesmilifene.

In an embodiment of the fifth aspect, the step of determining a value for P-gp expression comprises conducting an assay, wherein the assay is selected based on the cancer.

In an embodiment of the fifth aspect, the cancer is a leukemia or a lymphoma, and wherein the step of determining a value for P-gp expression comprises conducting a flow cytometry assay.

In an embodiment of the fifth aspect, the cancer is a leukemia or a lymphoma, and wherein the step of determining a value for P-gp expression comprises conducting a radiolabeled drug assay.

In an embodiment of the fifth aspect, the cancer is a leukemia or a lymphoma, and wherein the step of determining a value for P-gp expression comprises conducting a solid phase immunoassay that measures a cell-associated drug with an anti-drug antibody.

In an embodiment of the fifth aspect, the cancer is a solid tumor, and wherein the step of determining a value for P-gp expression comprises conducting an immunocytochemistry assay.

In an embodiment of the fifth aspect, the cancer is a solid tumor, and wherein the step of determining a value for P-gp expression comprises conducting an immunohistochemistry assay.

In an embodiment of the fifth aspect, the assay quantifies P-gp function.

In an embodiment of the fifth aspect, the cancer is acute myelogenous leukemia.

In an embodiment of the fifth aspect, the cancer is a carcinoma (e.g., breast cancer or ovarian cancer), a sarcoma, or a hematologic malignancy (e.g., acute lymphoblastic leukemia, chronic myeloid leukemia, plasma cell dyscrasias, lymphoma, or myelodysplasia).

In an embodiment of the fifth aspect, the chemotherapeutic agent is an anthracycline (e.g., doxorubicin, daunorubicin, epirubicin, idarubicin, or mitoxantrone).

In an embodiment of the fifth aspect, the chemotherapeutic agent is a Topoisomerase-II inhibitor (e.g., etoposide or teniposide).

In an embodiment of the fifth aspect, the chemotherapeutic agent is a vinca (e.g., vincristine, vinblastine, vinorelbine, or vindesine).

In an embodiment of the fifth aspect, the chemotherapeutic agent is a taxane (e.g., paclitaxel or docetaxel).

In an embodiment of the fifth aspect, the chemotherapeutic agent is Gleevec, dactinomycin, mitomycin, mithramycin, or Mylotarg.

### BRIEF DESCRIPTION OF THE DRAWINGS

FIG. 1 presents typical histograms of K562/R7 cells cultured in the absence of DiOC2, in the absence of zosuquidar, and in the presence of zosuquidar.

FIG. 2 provides a comparison between accumulation and efflux assays with leukemia cells.

FIG. 3 provides a comparison of efflux and accumulation assay data for leukemia cells in the absence or presence of zosuquidar.

FIG. 4 presents the efflux characteristics of K562/R7 cells loaded with 40 ng/ml DiOC2 in the presence of 200 ng/ml zosuquidar for 60 minutes.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

Cancer Targets

Many forms of cancer express P-gp, and thus can benefit from the administration of a P-gp efflux pump inhibitor when treated with a chemotherapeutic agent that is a substrate for P-gp efflux. For example, most solid tumors, lymphomas, bladder cancer, pancreatic cancer, ovarian cancer, liver cancer, myeloma, and sarcoma are all cancers with a P-gp expression of greater than 50%. Lymphocytic leukemia also has a P-gp expression of greater than 50%. The P-gp expression of breast cancers is about 30%. For metastatic breast cancer, 63% express P-gp. The methods and formulations of preferred embodiments are particularly efficacious in the treatment of any malignancy exhibiting some degree of P-gp expression and/or function, or in patients who are P-gp positive.

One form of cancer characterized by high rates of P-gp expression is acute myelogenous leukemia. There are approximately 11,000 new cases of AML per year in the United States and 9,000 new cases in the five major EU countries. In addition, the World Health Organization defines advanced myelodysplastic syndrome (MDS) as AML. There are approximately 4,000 cases of advanced MDS in the US and 3,000 cases in the five major EU countries. As a result, the target patient population for zosuquidar is approximately 15,000 patients in the U.S. and 12,000 in the major European markets.

Adult AML presents greater treatment challenges when compared to pediatric AML (age <15 years). Due in part to a more resilient patient population and a more sensitive disease, the 5 year survival rates for pediatric AML is 50% (late 1990s). In contrast, due in part to multiple co-morbid conditions and a more resistant disease, the 5 year survival rates for adult AML are only 13% (late 1990s). The 5 year survival rate for patients over 65 is only 7%.

Standard induction therapy in the U.S. for newly diagnosed AML patients is cytarabine with either idarubicin or daunorubicin (both P-gp substrates). In one study, 71% of AML patients greater than 60 years of age expressed moderate to high levels of P-gp. The expression was associated with a reduction in the complete remission (CR) rate. The CR rate for P-gp negative AML patients was 67% compared to 34% for P-gp positive patients. This combination of high levels of P-gp expression with the nearly universal use of drugs that are P-gp substrates provides a ready opportunity for the coadministration of a P-gp inhibitor in patients with AML.

Approximately 75% of AML patients are over age 60, and 71% are P-gp positive. The expression was associated with a reduction in the complete remission (CR) rate. The CR rate for P-gp negative AML patients was 67% compared to 34% for P-gp positive patients. Clinical outcomes in terms of patient survival rates are significantly better for patients that are P-gp negative than for those that are P-gp positive—a 50% survival rate at approximately 3-4 months for P-gp positive patients, versus a 50% survival rate at approximately 15 months for P-gp negative patients. See Campos, et al., Blood, 79:473-476, 1992.

Approximately 75% of AML patients will eventually relapse and be candidates for additional treatment. Relapsed AML patients typically require prolonged hospitalization, and their prognosis is generally poor. Of these relapsed patients, approximately 80% are P-gp positive.

Over 129,000 metastatic breast cancer patients are treated with chemotherapy in the United States and Europe annually. Of these patients, over 81,000 are treated with a P-gp substrate. 41% of breast cancers express P-gp. For metastatic breast cancer, 63% express P-gp.

Zosuquidar

U.S. Pat. Nos. 5,643,909 and 5,654,304 disclose a series of 10,11-methanobenzosuberane derivatives useful in enhancing the efficacy of existing cancer chemotherapeutics and for treating multidrug resistance. One such derivative having good activity, oral bioavailability, and stability, is zosuquidar, a compound of formula (2R)-anti-5-3-[4-(10,11-difluoromethanodibenzosuber-5-yl)piperazin-1-yl]-2-hydroxypropoxy}quinoline.

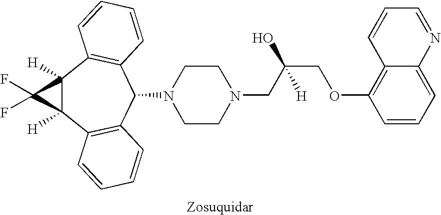

Given the limitations of previous generations of MDR modulators, three preclinical critical success factors were identified and met for zosuquidar: 1) it is a potent inhibitor of P-glycoprotein; 2) it is selective for P-glycoprotein; and 3) no pharmacokinetic interaction with co-administered chemotherapy is observed.

Zosuquidar is extremely potent in vitro (Kᵢ=59 nM) and is among the most active modulators of P-gp-associated resistance described to date. Zosuquidar has also demonstrated good in vivo activity in preclinical animal studies. In addition, the compound does not appear to be a substrate for P-gp efflux, resulting in a relatively long duration of reversal activity in resistant cells even after the modulator has been withdrawn.

Another significant attribute of zosuquidar as an MDR modulator is the minimal pharmacokinetic (PK) interactions with several oncolytics tested in preclinical models. Such minimal PK interaction permits normal doses of oncolytics to be administered and also a more straightforward interpretation of the clinical results.

Assays for Multidrug Resistance

There are a variety of techniques to detect expression of MDR1. The detection and quantitation of MDR1 protein is typically achieve using immunological techniques. For hematopoietic cells such as those from leukemia or lymphoma patients, the techniques include flow cytometry and fixed cells on microscope slides. The cells are treated with antibodies specific for the MDR1 protein, such as the mouse ARK-16 monoclonal antibody. Such antibodies can be directly labeled with fluorescent probe, or detected using subsequent reagents such as goat anti-mouse IgG-FITC. Flow cytometry allows for direct quantitative determinations of the full spectrum of MDR1 expression using channel number or fluorescence intensity. Microscopic examination of the slide preparations can give qualitative results (−, +, ++, and the like) or, in conjunction with an image analyzer, quantitative evaluations typically expressed in pixels.

For solid tumors, such as breast cancer, typically immunocytochemistry (ICC) or immunohistochemistry (IHC) techniques are employed. Using, for example, frozen sections or paraffin blocks, the detection techniques are the same as described for fixed leukemia cells on microscope slides. Expression of MDR1 can also be achieved with the measurement of specific mRNA levels. Cell slides can be processed, and levels of mRNA discerned using basic molecular biology techniques such as quantitative fluorescent PCR. Alternatively, the cells of interest can be lysed, processed, and following PCR of the mRNA, the product can be detected and quantitated following gel electrophoresis. Anti-sense targeting of MDR1 mRNA is also possible, followed by standard techniques for quantitative determinations. Radio-labeled probes followed by autoradiography or other radiodetection techniques can also be used to get a relative estimate of MDR1 protein or mRNA expression. Thus, there exists a broad range of methods for the detection and quantitation of the spectrum of MDR1 expression exhibited by a patient population.

The relative expression of MDR1 is possible in vivo. MDR1-specific antibodies labeled with any number of detectable markers, such as radioactive compounds detectable with positron emission tomography (PET), single-photon emission computed tomography (SPECT) or compounds detectable with magnetic resonance imaging (MRI) can be used to assess MDR1 expression in patients with cancer.

Of equal importance to MDR1 protein and mRNA expression is the quantitation of MDR1 function, i.e., functional expression. MDR1 functions as a cytoplasmic membrane pump, effluxing compounds such as drugs and toxins from the cytoplasm to the exterior of the cell. Compounds acted on by MDR1 are termed MDR1 substrates. Detection of MDR1 function therefore involves detection of substrate efflux, such as drugs or, alternatively, detecting efflux of surrogate fluorescent dye markers as DiOC2 (3,3′-diethyloxacarbocyanine iodide) or Rhodamine 123 (Rh123, or 2-(6-amino-3-imino-3H-xanthen-9-yl)benzoic acid, methyl ester). For single cell suspensions, such as blood or bone marrow from leukemia patients, the cells are exposed in tissue culture to a substrate for MDR1, such as the aforementioned dye markers, radiolabeled drugs, or drugs that can be quantitated by other means such as fluorescence. At physiological temperature (37° C.) the net accumulation of the substrate over time, in the presence or absence of specific MDR1 inhibitors, gives an indication of the MDR1 functional activity exhibited by the cells. Alternatively, the single cell suspension can be exposed to the substrate, i.e., loaded with, and subsequent efflux of the substrate over time monitored at physiological temperature in the presence or absence of specific MDR1 inhibitors.

PET, SPECT, and MRI techniques can also be used to assess MDR1 function in cancer patients. Thus, small organic chemicals as well as metal complexes which serve as MDR1 substrates can be rendered as radionuclides or other markers which are detectable by the imaging technologies. Additionally, functional expression in solid tumors can be more efficiently ascertained by ICC/IHC techniques with prior labeling of the tumor cells while in the patient.

Diagnostic Testing for P-gp Expression and Efflux Pump Activity

Diagnostic testing methods for P-gp expression and efflux pump activity can be used to prospectively stratify patients for treatment optimization in treating malignancies exhibiting P-gp expression or function, such as acute myelogenous leukemia, most solid tumors, lymphomas, bladder cancer, pancreatic cancer, ovarian cancer, liver cancer, myeloma, lymphocytic leukemia, and sarcoma.

In a particularly preferred embodiment, susceptibility of the individual patient's cancer and natural kill (NK) cells to the inhibitory effects of zosuquidar on that patient's P-gp expression and function is incorporated into the treatment regimen. Thus, the cells of interest are assessed as in one of the above-described methods both before and after exposure to zosuquidar. Such an assay can be considered a “zosuquidar drugability” assay to determine the potential for improved response to chemotherapy with administration of zosuquidar. Similar methodology can be employed in providing assays to determine drugability for other P-gp efflux inhibitors, and thus determine the potential for improved response to chemotherapy treatment regimens employing P-gp efflux inhibitors.

Ranges of P-gp efflux pump activation and populations of cells have been correlated to identify and stratify patients for future treatment with enhanced clinical outcomes. The diagnostic testing employed preferably includes assay methods as described above, including immunophenotyping and cytogenetics of the cancer cells, as well as a diagnostic algorithm to relate the phenotypic and clinical data to chemotherapeutic response potential.

While the techniques are especially preferred for use in administering zosuquidar, the techniques are applicable to any therapeutic drug that is a substrate for P-gp efflux. Such drugs include, but are not limited to, P-glycoprotein substrates; anticancer drugs (e.g., vinca alkaloids such as vinblastine and vincristine; anthracyclines such as doxorubicin, daunorubicin, epirubicin; anthracenes such as bisantrene and mitoxantrone; epipodophyllo-toxins such as etoposide and teniposide; and other anticancer drugs such as actinomyocin D, mithomycin C, mitramycin, methotrexate, docetaxel, etoposide (VP-16), paclitaxel, docetaxel, and adriamycin); immunosuppressants (e.g., cyclosporine A, tacrolimus); steroids (e.g., dexamethasone, hydrocortisone, corticosterone, triamcinolone, aldosterone, methylprednisolone); antiepileptics (e.g., phenytoin); antidepressants (e.g., citalopram, thioperidone, trazodone, trimipramine, amitriptyline, phenothiazines); antipsychotics (fluphenazine, haloperidol, thioridazine, trimipramine); HIV protease inhibitors (e.g., amprenavir, indinavir, lopinavir, nelfinavir, ritonavir, saquinavir); calcium blockers (e.g., bepridil, diltiazem, flunarizine, lomerizine, secoverine, tamolarizine, verapamil, nicardipine, prenylamine, fendiline); and cardiac drugs (e.g., digoxin, diltiazem, verapamil, talinolol).

A P-gp diagnostic test exhibits clinical utility for a broad range of disease states. As discussed above, the drugs which are substrates of P-gp are quite varied as are the associated disease states. The use of a P-gp diagnostic test is best exemplified in the treatment of AML, where it has been demonstrated that the levels of P-gp expression and function are significantly related to response to the chemotherapy.

Preferred embodiments involve assessment on a patient-specific basis for zosuquidar to modulate P-gp function in the cancer cell population. Thus, the expression and function of P-gp is assessed in the absence and presence of zosuquidar with the patient's cancer and NK cells. A statistical algorithm to relate immunophenotype of the cancer cells, P-gp function and expression, cytogenetics, and certain clinical variables such as age to response potential to chemotherapy is used. This methodology can be applied to any form of cancer as related to chemotherapeutic agents which are substrates of P-gp as listed above.

The distribution of P-gp expression also has utility for a P-gp diagnostic beyond the oncology field. Affected organs can include the intestine, liver, placenta, kidneys, and blood brain barrier (BBB). The system of P-gp expression is believed to have evolved to eliminate or exclude toxins and metabolites. For unknown reasons, hematopoietic and lymphoid cells, such as myeloid, NK, and CD8 (cytotoxic T cells, CTL) cells also express P-gp. Disease states originating from these organs/cells can exhibit varied responses to therapy depending on the levels of P-gp expression. Moreover, P-gp is highly expressed by mucosal and luminal surfaces of organs which are involved in drug absorption, distribution, and excretion. Varied expression of P-gp can influence therapy by influencing drug pharmacokinetics in addition to pharmacodynamics. Therefore, a P-gp diagnostic test capable of stratifying patient populations according to therapeutic response potential has substantial clinical utility for a variety of disease conditions.

For example, treatment of autoimmune, other inflammatory diseases, and organ transplant with immunosuppressive drugs, as well as with steroids, can be optimized using a P-gp diagnostic test. This is indicated by the enhanced expression of MDR1 in renal transplant patients who undergo graft rejection on cyclosporine. Other examples include patients with rheumatoid arthritis, inflammatory bowel disease, and systemic lupus erythematosus. Varied expression of P-gp by the BBB can predict response to epilepsy drugs and other drugs used to treat mental disorders. Response potential to treatment of HIV and other viral therapeutics can also be optimized with a P-gp diagnostic test. Such a test can also find clinical utility in the prediction of adverse events, especially as related to the use of cardiovascular drugs. Such a test is not currently being conducted to predict prospectively response to therapy for this diverse disease set.

The identification of tumor cells expressing P-gp or exhibiting positive P-gp function involves a relationship between negative and positive control situations. Expression assays that use antibodies to detect P-gp expression include controls of cells either not treated with primary antibody, or cells treated with an antibody of the same isotype as that of the anti-P-gp reagent. At a cutoff where cells treated under these latter conditions exhibited minimal positive reaction, e.g., 0-5% positive cells, positive results using the anti-P-gp antibody include 10% or more positive cells, typically 10-25% positive cells. Functional assays involve a ratio of P-gp dye accumulation or efflux in the presence and absence of a specific P-gp inhibitor such as zosuquidar. For the accumulation assay format the amount of dye taken up by the cells in the presence of zosuquidar is divided by that amount of dye taken up by cells cultured without zosuquidar. For the efflux assay format the amount of dye eliminated from the cells in the presence of zosuquidar is divided by that amount of dye eliminated without zosuquidar in the culture. Positive P-gp functional activity is inferred for ratios greater than about 1:1.2, typically ratios of from about 1:1.2 to about 1:1.5. For the efflux P-gp functional assay, ratios can be used, as well as % efflux and % inhibition of efflux by zosuquidar or other P-gp modulators. Positive P-gp functional activity is inferred by % efflux of from about 30% to about 50%, and specific inhibition by a P-gp modulator is similarly from about 30% to about 50%.

Chemotherapeutic Regimens Utilizing Zosuquidar and Mylotarg

In preferred embodiments, a P-gp expression or efflux pump activity diagnostic is conducted to provide information in treating AML patients or patients with metastatic breast cancer with zosuquidar in combination with Mylotarg. If the results of the P-gp expression or efflux pump activity diagnostic indicates positive P-gp expression or efflux pump activity, then treatment with zosuquidar (or another P-gp efflux inhibitor) in combination with Mylotarg is initiated. If the results of the P-gp expression or efflux pump activity diagnostic indicate negative P-gp expression or efflux pump activity, then zosuquidar is expected not to yield an improvement in clinical outcome and another treatment option not involving administration of a P-gp efflux inhibitor is selected. In relapsed AML patients, it is generally considered acceptable clinical practice to wait for P-gp expression or efflux pump activity test results before initiating a treatment. However, in certain embodiments it can be desirable to initiate treatment before receiving test results, and then reevaluate the desirability of continuing treatment, depending upon the test results. Most preferably, P-gp expression or efflux pump activity of a sample both in the presence and absence of the P-gp efflux inhibitor is compared, whereby the P-gp efflux that is inhibitable by the P-gp efflux inhibitor can be determined. However, in certain embodiments wherein P-gp expression or function status correlates with expectation of clinical success, it can be useful to determine P-gp expression or efflux pump activity at any point in time.

Mylotarg was approved in May 2000 for relapsed CD33-positive AML patients over the age of 60. Mylotarg from Wyeth and Celltech is based on antibody-targeted chemotherapy. Mylotarg's highly specific antibody recognizes a cell-surface molecule, CD33, which is abundant on AML cells (>90%) but absent from normal blood stem cells, the seeds from which normal blood and immune cells originate. The antibody is linked to calicheamicin, a potent chemotherapy agent. The antibody selectively targets leukemic blast cells and delivers calicheamicin to them. The chemical structure of Mylotarg is provided below.

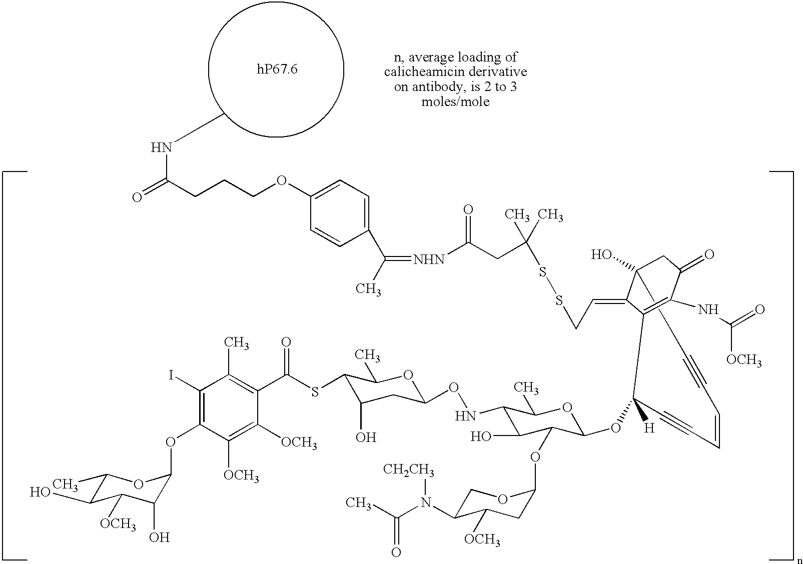

There is a growing body of evidence to suggest that the calicheamicin component of Mylotarg is also an MDR substrate and subject to the P-gp efflux pump. In several studies, the cytotoxic effect of Mylotarg has been shown to be inversely correlated with the amount of P-gp present. Two MDR modulators, valspodar and the quinolone derivative MS-209, have both been shown to reverse the resistance to Mylotarg in P-gp expressing CD33(+) leukemia cells and clinical studies are underway in combination with cyclosporine.

The combination of zosuquidar, a highly specific and safe P-gp efflux inhibitor, in combination with Mylotarg or another calicheamicin-antibody conjugate is effective for treatment of relapsed AML. The effective dose of zosuquidar and the timing of administration of zosuquidar and Mylotarg are critical to achieving improved complete remission rates and enhanced leukemia free and overall survival rates in the relapsed AML patient population. While the methods and formulations of preferred embodiments are especially preferred for treatment of relapsed AML patients, the methods and formulations can be adapted to other drugs and indications. For example, P-gp efflux inhibitors other than zosuquidar and/or chemotherapeutics other than Mylotarg can be administered according to the disclosed dosing regimens, or slightly modified dosing regimens. Likewise, the formulations and dosing regimens employing zosuquidar and Mylotarg can be employed in treating AML patients other than relapsed AML patients, or for other types of leukemia or other cancers that express P-gp, e.g., many solid tumors, lymphomas, bladder cancer, pancreatic cancer, ovarian cancer, liver cancer, myeloma, lymphocytic leukemia, breast cancer, and sarcoma.

Zosuquidar or certain other therapeutic agents can be administered in the form of a pharmaceutically acceptable salt, e.g., the trihydrochloride salt. The terms “pharmaceutically acceptable salts” and “a pharmaceutically acceptable salt thereof” as used herein are broad terms and are used in their ordinary sense, including, without limitation, to refer to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of zosuquidar or other therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, and protected derivatives can also be suitable for use in compositions and methods of preferred embodiments. Also suitable for administration are selected therapeutic agents in hydrated form, selected enantiomeric forms of certain therapeutic agents, racemic mixtures of certain therapeutic agents, and the like.

Contemplated routes of administration include topical, oral, subcutaneous, parenteral, intradermal, intramuscular, intraperitoneal, and intravenous. However, it is particularly preferred to administer zosuquidar and/or Mylotarg in intravenous form. The combination or individual components can be in any suitable solid or liquid form. A particularly preferred form comprises a lyophilized form that is reconstituted for intravenous administration.

Zosuquidar and/or other therapeutic agents can be formulated into liquid preparations for, e.g., oral, nasal, anal, rectal, buccal, vaginal, peroral, intragastric, mucosal, perlingual, alveolar, gingival, olfactory, or respiratory mucosa administration. Suitable forms for such administration include suspensions, syrups, and elixirs. If nasal or respiratory (mucosal) administration is desired (e.g., aerosol inhalation or insufflation), compositions may be in a form and dispensed by a squeeze spray dispenser, pump dispenser or aerosol dispenser. Aerosols are usually under pressure by means of a hydrocarbon. Pump dispensers can preferably dispense a metered dose or a dose having a particular particle size.

The pharmaceutical compositions containing zosuquidar and/or other therapeutic agents are preferably isotonic with the blood or other body fluid of the patient. The isotonicity of the compositions can be attained using sodium tartrate, propylene glycol or other inorganic or organic solutes. Sodium chloride is particularly preferred. Buffering agents can be employed, such as acetic acid and salts thereof, citric acid and salts thereof, boric acid and salts thereof, and phosphoric acid and salts thereof. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like.

Viscosity of the pharmaceutical compositions can be maintained at a selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener can depend upon the thickening agent selected. An amount is preferably used that can achieve the selected viscosity. Viscous compositions are normally prepared from solutions by the addition of such thickening agents.

A pharmaceutically acceptable preservative can be employed to increase the shelf life of the pharmaceutical compositions. Benzyl alcohol can be suitable, although a variety of preservatives including, for example, parabens, thimerosal, chlorobutanol, and benzalkonium chloride can also be employed. A suitable concentration of the preservative is typically from about 0.02% to about 2% based on the total weight of the composition, although larger or smaller amounts can be desirable depending upon the agent selected.

The zosuquidar and/or other therapeutic agents can be in admixture with a suitable carrier, diluent, or excipient such as sterile water, physiological saline, glucose, and the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., standards texts such as “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

For oral administration, the zosuquidar and/or other therapeutic agents can be provided as a tablet, aqueous or oil suspension, dispersible powder or granule, emulsion, hard or soft capsule, syrup, or elixir. Compositions intended for oral administration can be prepared according to any method known in the art for the manufacture of pharmaceutical compositions and can include one or more of the following agents: sweeteners, flavoring agents, coloring agents and preservatives. Aqueous suspensions can contain the active ingredient in admixture with excipients suitable for the manufacture of aqueous suspensions.

Formulations for oral administration can also be provided as hard gelatin capsules, wherein the zosuquidar and/or other active ingredients are mixed with an inert solid diluent, such as calcium carbonate, calcium phosphate, or kaolin, or as soft gelatin capsules. In soft capsules, the active ingredients can be dissolved or suspended in suitable liquids, such as water or an oil medium, such as peanut oil, olive oil, fatty oils, liquid paraffin, or liquid polyethylene glycols. Stabilizers and microspheres formulated for oral administration can also be used. Capsules can include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc and magnesium stearate and, optionally, stabilizers.

Tablets can be uncoated or coated by known methods to delay disintegration and absorption in the gastrointestinal tract and thereby provide a sustained action over a longer period of time. For example, a time delay material such as glyceryl monostearate can be used. When administered in solid form, such as tablet form, the solid form typically comprises from about 0.001 wt. % or less to about 50 wt. % or more of active ingredient(s) including zosuquidar and/or other therapeutic agents, preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets can contain the zosuquidar and/or other therapeutic agents in admixture with non-toxic pharmaceutically acceptable excipients including inert materials. For example, a tablet can be prepared by compression or molding, optionally, with one or more additional ingredients. Compressed tablets can be prepared by compressing in a suitable machine the active ingredients in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Preferably, each tablet or capsule contains from about 10 mg or less to about 1,000 mg or more of each of zosuquidar and/or other therapeutic agents, more preferably from about 20, 30, 40, 50, 60, 70, 80, 90, or 100 mg to about 150, 200, 250, 300, 350, 400, 450, 500, 550, 600, 650, 700, 750, 800, or 900 mg. Most preferably, tablets or capsules are provided in a range of dosages to permit divided dosages to be administered. A dosage appropriate to the patient and the number of doses to be administered daily can thus be conveniently selected. While in certain embodiments it can be preferred to incorporate the zosuquidar and any other therapeutic agent employed in combination therewith in a single tablet or other dosage form, in certain embodiments it can be desirable to provide the zosuquidar and other therapeutic agents in separate dosage forms, e.g., zosuquidar in a dosage form separate from other agents(s). Combinations of dosage forms can also be employed, e.g., oral and,intravenous.

Suitable inert materials include diluents, such as carbohydrates, mannitol, lactose, anhydrous lactose, cellulose, sucrose, modified dextrans, starch, and the like, and inorganic salts such as calcium triphosphate, calcium phosphate, sodium phosphate, calcium carbonate, sodium carbonate, magnesium carbonate, and sodium chloride. Disintegrants or granulating agents can be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya, and tragacanth, and alginic acid and salts thereof.

Binders can be used to form a hard tablet. Binders include materials from natural products such as acacia, tragacanth, starch, gelatin, methyl cellulose, ethyl cellulose, carboxymethyl cellulose, polyvinyl pyrrolidone, hydroxypropylmethyl cellulose, and the like.

Lubricants, such as stearic acid and magnesium or calcium salts thereof, polytetrafluoroethylene, liquid paraffin, vegetable oils, waxes, sodium lauryl sulfate, magnesium lauryl sulfate, polyethylene glycol, starch, talc, pyrogenic silica, hydrated silicoaluminate, and the like can be included in tablet formulations.

Surfactants can also be employed, for example, anionic detergents such as sodium lauryl sulfate, dioctyl sodium sulfosuccinate, and dioctyl sodium sulfonate, cationic detergents such as benzalkonium chloride and benzethonium chloride, and/or nonionic detergents such as polyoxyethylene hydrogenated castor oil, glycerol monostearate, polysorbates, sucrose fatty acid ester, methyl cellulose, and carboxymethyl cellulose.

Controlled-release formulations can be employed wherein the zosuquidar and/or other therapeutic agents are incorporated into an inert matrix that permits release by either diffusion or leaching mechanisms. Slowly degenerating matrices can also be incorporated into the formulation. Other delivery systems can include timed release, delayed release, or sustained release delivery systems. Nanoparticulate systems or nanoparticulate forms of the active ingredients can advantageously be employed in certain embodiments.

Coatings can be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone, polyethylene glycols, and enteric materials such as phthalic acid esters. Dyestuffs and pigments can be added for identification or to characterize different combinations of active compound doses

When administered orally in liquid form, a liquid carrier such as water, petroleum, oils of animal or plant origin such as peanut oil, mineral oil, soybean oil, or sesame oil, or synthetic oils can be added to the zosuquidar and/or other therapeutic agents. Physiological saline solution, dextrose, other saccharide solutions, and glycols such as ethylene glycol, propylene glycol, and polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents.

Pulmonary delivery of zosuquidar and/or other therapeutic agents can also be employed. The zosuquidar and/or other therapeutic agents are delivered to the lungs while inhaling and traverse across the lung epithelial lining to the blood stream. A wide range of mechanical devices designed for pulmonary delivery of therapeutic products can be employed, including but not limited to nebulizers, metered dose inhalers, and powder inhalers, all of which are familiar to those skilled in the art. These devices employ formulations suitable for the dispensing of zosuquidar and/or other therapeutic agents. Typically, each formulation is specific to the type of device employed and can involve the use of an appropriate propellant material, in addition to diluents, adjuvants, and/or carriers useful in therapy.

The zosuquidar and/or other therapeutic agents are advantageously prepared for pulmonary delivery in particulate form with an average particle size of from 0.1 m or less to 10 μm or more, more preferably from about 0.2, 0.3, 0.4, 0.5, 0.6, 0.7, 0.8, or 0.9 μm to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 μm. Pharmaceutically acceptable carriers for pulmonary delivery of zosuquidar and/or other therapeutic agents include carbohydrates such as trehalose, mannitol, xylitol, sucrose, lactose, and sorbitol. Other ingredients for use in formulations can include dipalmitoylphosphatidylcholine (DPPC), 1,2-sn-dioleoylphosphatidylcholine (DOPE), disteroylphosphatidylcholine (DSPC), and dioleoylphosphatidyl-choline (DOPC). Natural or synthetic surfactants can be used, including polyethylene glycol and dextrans, such as cyclodextran. Bile salts and other related enhancers, as well as cellulose and cellulose derivatives, and amino acids can also be used. Liposomes, microcapsules, microspheres, inclusion complexes, and other types of carriers can also be employed.

Pharmaceutical formulations suitable for use with a nebulizer, either jet or ultrasonic, typically comprise the zosuquidar and/or other therapeutic agents dissolved or suspended in water at a concentration of about 0.01 mg or less to 100 mg or more of zosuquidar and/or other therapeutic agents per mL of solution, preferably from about 0.1, 1, 2, 3, 4, 5, 6, 7, 8, 9, or 10 mg per mL of solution to about 15, 20, 25, 30, 35, 40, 45, 50, 55, 60, 65, 70, 75, 80, 85, or 90 mg per mL of solution. The formulation can also include a buffer and a simple sugar (e.g., for protein stabilization and regulation of osmotic pressure). The nebulizer formulation can also contain a surfactant, to reduce or prevent surface induced aggregation of the zosuquidar and/or other therapeutic agents caused by atomization of the solution in forming the aerosol.

Formulations for use with a metered-dose inhaler device generally comprise a finely divided powder containing the active ingredients suspended in a propellant with the aid of a surfactant. The propellant can include conventional propellants, such as chlorofluorocarbons, hydrochlorofluorocarbons, hydrofluorocarbons, and hydrocarbons. Preferred propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations suitable for dispensing from a powder inhaler device typically comprise a finely divided dry powder containing zosuquidar and/or other therapeutic agents, optionally including a bulking agent, such as lactose, sorbitol, sucrose, mannitol, trehalose, or xylitol in an amount that facilitates dispersal of the powder from the device, typically from about 1 wt. % or less to 99 wt. % or more of the formulation, preferably from about 5, 10, 15, 20, 25, 30, 35, 40, 45, or 50 wt. % to about 55, 60, 65, 70, 75, 80, 85, or 90 wt. % of the formulation.

When zosuquidar and/or other therapeutic agents are administered by intravenous, cutaneous, subcutaneous, parenteral, or other injection, they are preferably in the form of pyrogen-free, parenterally acceptable aqueous solution or oleaginous suspension. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred pharmaceutical composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed, including synthetic monoglycerides and diglycerides. In addition, fatty acids such as oleic acid can likewise be used in the formation of injectable preparations. The pharmaceutical compositions can also contain stabilizers, preservatives, buffers, antioxidants, and other additives known to those of skill in the art.

The duration of the injection can be adjusted depending upon various factors, and can comprise a single injection administered over the course of a few seconds or less to 1, 2, 3, 4, 5, 6, 7, 8, 9, 10, 11, 12, 13, 14, 15, 16, 17, 18, 19, 20, 21, 22, 23, 24, 26, 28, 30, 32, 34, 36, 40, 44, 48, 54, 60, 66, 72, 78, 84, 90, or 96 hours or more of continuous intravenous administration.

The zosuquidar and/or other therapeutic agents can be administered systemically or locally, via a liquid or gel, or as an implant or device.

The compositions of the preferred embodiments can additionally employ adjunct components conventionally found in pharmaceutical compositions in their art-established fashion and at their art-established levels. Thus, for example, the compositions can contain additional compatible pharmaceutically active materials for combination therapy (such as supplemental P-gp inhibitors, chemotherapeutic agents, and the like), or can contain materials useful in physically formulating various dosage forms of the preferred embodiments, such as excipients, dyes, perfumes, thickening agents, stabilizers, preservatives and antioxidants.

The zosuquidar and/or other therapeutic agents can be provided to an administering physician or other health care professional in the form of a kit. The kit is a package which houses one or more containers which contain zosuquidar and/or additional therapeutic agents in suitable form and instructions for administering the pharmaceutical composition to a subject. The kit can optionally also contain one or more additional therapeutic agents. The kit can optionally contain one or more assays or diagnostic tools and instructions for use, e.g., a diagnostic to measure efflux pump activity or P-gp expression or function. For example, a kit containing a single composition comprising zosuquidar with one or more chemotherapeutic agents can be provided, or separate pharmaceutical compositions containing zosuquidar and other therapeutic agents can be provided. The kit can also contain separate doses of zosuquidar and/or other therapeutic agents for serial or sequential administration. The kit can contain suitable delivery devices, e.g., syringes, inhalation devices, and the like, along with instructions for administrating zosuquidar and/or other therapeutic agent. The kit can optionally contain instructions for storage, reconstitution (if applicable, e.g., for a lyophilized form reconstituted for intravenous administration), and administration of any or all therapeutic agents included. The kits can include a plurality of containers reflecting the number of administrations to be given to a subject. In a particularly preferred embodiment, a kit for the treatment of AML is provided that includes both zosuquidar, a chemotherapeutic agent, and one or more diagnostics or instructions for conducting one or more diagnostics for determining P-gp expression and/or efflux pump activity. In a particularly preferred embodiment, the kit includes a “zosuquidar drugable” assay, as previously described.

Zosuquidar and a therapeutic agent that is a substrate for P-gp efflux can be administered to patients suffering from AML prior to confirmation of P-gp expression or function, or to AML patients other than relapse AML patients. However, such therapy is preferably administered to relapsed AML patients. The administration route, amount administered, and frequency of administration can vary depending on the age of the patient, status as relapsed or newly diagnosed AML patient, and severity of the condition.

Contemplated amounts of Mylotarg for intravenous administration to treat relapsed AML are from about 10 mg/day or less to about 1000 mg/day or more administered on one, two, or more separate days. The dosage is preferably administered intravenously at a rate of about 1 mg/m² or less to about 10 mg/m² or more continuously over the course of about 2, 3, or 4 hours to about 5, 6, 7, 8, 9, 10, 11, 12, 13, 14, 15, 16, 17, 18, 19, 20, 21, 22, 23, or 24 hours, more preferably over the course of about 2 hours to about 6 hours; however, administration at a rate of 5 mg/m², 7 mg/m², or 9 mg/m² over about 2 hours is particularly preferred. Preferably, doses of Mylotarg are administered on Day 1 and Day 15 of the treatment regimen. However, in certain embodiments, the second dose can be administered on Day 5, 6, 7, 8, 9, 10, 11, 12, 13, 14, 16, 17, 18, 19, 20, 21, or 22, or another day of the treatment regimen. Other dosing regimens include administering three doses total over a week.

Contemplated amounts of zosuquidar for intravenous administration to treat relapsed AML are from about 400 mg/day or less to about 1,600 mg/day or more, preferably from about 500, 600, or 700 mg/day to about 900, 1000, 1100, 1200, 1300, 1400, or 1500 mg/day, and most preferably from about 500 mg/day to about 800 mg/day. It is generally preferred to start the infusion of zosuquidar from about 2 hours or less to about 6 hours or more prior to the administration of Mylotarg. In the course of a treatment regimen, the zosuquidar is preferably administered on two, three, or four separate days. The dosage is preferably administered in intravenously continuously over the course of about 6 to 90 hours, more preferably over the course of 12, 18, 24, 30, 36, or 42 hours to about 54, 60, 66, 72, 78, or 84 hours, most preferably over about 24 hours, 48 hours, or 72 hours, depending upon the treatment regimen. Preferably the zosuquidar is administered on Day 1 of the treatment regimen. In certain embodiments, additional zosuquidar is administered on Day 2, on Days 2 and 3, or on Days 2, 15, and 16. However, in certain embodiments, one, two, or three or more additional doses can be administered on other days of the treatment regimen.

Table 1 provides various dosing regimes that can be used in treating relapsed AML.

TABLE 1

| Dose | | |
|---|---|---|
| Level | Mylotarg | Zosuquidar |
| −1* | 5 mg/m² IV over 4 hr Day 1 and 15 | 800 mg/day continuous |
| | | IV over 24 hr |
| | | Day 1 and 15 |
| 1 | 5 mg/m² IV over 4 hr Day 1 and 15 | 800 mg/day continuous |
| | | IV over 48 hr |
| | | Day 1&2 and 15&16 |
| 2 | 7 mg/m² IV over 4 hr Day 1 and 15 | 800 mg/day continuous |
| | | IV over 48 hr |
| | | Day 1&2 and 15&16 |
| 3 | 9 mg/m² IV over 4 hr Day 1 and 15 | 800 mg/day continuous |
| | | IV over 48 hr |
| | | Day 1&2 and 15&16 |
| 4 | 9 mg/m² IV over 4 hr Day 1 and 15 | 800 mg/day continuous |
| | | IV over 72 hr |
| | | Day 1-3 and 15-17 |

*Only if level 1 has a dose limiting toxicity (DLT).

Table 2 provides alternative dosing regimes that can be used in treating relapsed AML.

TABLE 2

| Dose | | |
|---|---|---|
| Level | Mylotarg | Zosuquidar |
| −1* | 5 mg/m² IV over 6-24 | 500-700 mg/day continuous IV over 24 hr |
| | hr Day 1 and 15 | Day 1 and 15 |
| 1 | 5 mg/m² IV over 6-24 | 500-700 mg/day continuous IV over 48 hr |
| | hr Day 1 and 15 | Day 1&2 and 15&16 |
| 2 | 7 mg/m² IV over 6-24 | 500-700 mg/day continuous IV over 48 hr |
| | hr Day 1 and 15 | Day 1&2 and 15&16 |
| 3 | 9 mg/m² IV over 6-24 | 500-700 mg/day continuous IV over 48 hr |
| | hr Day 1 and 15 | Day 1&2 and 15&16 |
| 4 | 9 mg/m² IV over 6-24 | 500-700 mg/day continuous IV over 72 hr |
| | hr Day 1 and 15 | Day 1-3 and 15-17 |

*Only if level 1 has a dose limiting toxicity (DLT).

A clinical study was conducted to determine the efficacy of Mylotarg in the treatment of relapsed AML. It was determined that the rate of complete remission (CR+CRp) for P-gp negative patients treated with Mylotarg was 64% (N=36). In contrast, the rate of complete remission for P-gp positive patients was only 9% (N=22). This indicates that P-gp efflux plays an important role in survival rates for relapsed AML, and further indicates that inhibition of P-gp efflux, e.g., by also administering zosuquidar or another P-gp efflux inhibitor, has the potential to significantly improve response rates in P-gp positive patients. The diagnostic and assay methods described herein are therefore useful in treating relapsed AML. Likewise, a diagnostic or assay to determine P-gp expression or function or efflux pump activity can be useful in devising treatment regimens for other cancers, such as metastatic breast cancer, that also exhibit P-gp expression.

Chemotherapeutic Regimens Utilizing Zosuquidar, Daunorubicin, and Cytarabine

In preferred embodiments, a P-gp expression or efflux pump activity diagnostic is conducted to provide information in treating newly diagnosed AML patients with zosuquidar in combination with daunorubicin and cytarabine. In newly diagnosed AML patients, it is generally not considered acceptable clinical practice to wait for P-gp expression or efflux pump activity test results before initiating a treatment. Accordingly, treatment is initiated immediately after diagnosis. When test results become available, the desirability of continuing treatment can be evaluated, depending upon the test results. Typically, when the results of the P-gp expression or efflux pump activity diagnostic indicate negative P-gp expression, then treatment with a P-gp efflux inhibitor is discontinued because administration of the drug is not expected to contribute to an improved clinical outcome. Preferably, P-gp expression or function or efflux pump activity is determined both in the presence and the absence the P-gp efflux inhibitor to determine the P-gp expression that is inhibitable by the P-gp efflux inhibitor.

Daunorubicin is an antibiotic chemotherapy treatment that is widely used to treat acute myeloid leukemia and acute lymphocytic leukemia. It is produced by the bacteria Streptomyces coeruleorubidis and was approved by the FDA as a first line therapy treatment for leukemia in 1998. Daunorubicin is typically administered intravenously. It is marketed under the brand names Cerubidine, DaunoXome, and Liposomal daunorubicin. Daunorubicin has the following structure:

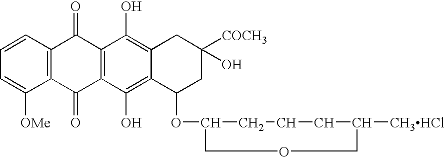

Cytarabine is a deoxycytidine analogue, cytosine arabinoside (ara-C), which is metabolically activated to the triphosphate nucleotide (ara-CTP), which acts as a competitive inhibitor of DNA polymerase and produces S phase-specific cytotoxicity. It is used as an antineoplastic, generally as part of a combination chemotherapy regimen, in the treatment of acute lymphocytic and acute myelogenous leukemia, the blast phase of chronic myelogenous leukemia, erythroleukemia, and non-Hodgkin's lymphoma. It is typically administered intravenously and subcutaneously, and for the prophylaxis and treatment of meningeal leukemia, administered intrathecally. Cytarabine has the following structure:

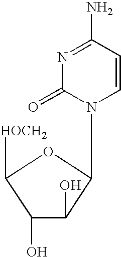

The combination of zosuquidar, the antibiotic chemotherapeutic daunorubicin, and the antineoplastic cytarabine, is effective for treatment of newly diagnosed AML. The effective dose of zosuquidar and the timing of administration of zosuquidar, daunorubicin, and cytarabine are critical to achieving improved complete remission rates and enhanced leukemia free survival rates in the newly diagnosed AML patient population. While the methods and formulations of preferred embodiments are especially preferred for treatment of newly diagnosed AML patients, the methods and formulations can be adapted to other drugs and indications. For example, P-gp efflux inhibitors other than zosuquidar and/or chemotherapeutics other than daunorubicin and cytarabine can be administered according to the disclosed dosing regimens, or slightly modified dosing regimens. Likewise, the formulations and dosing regimens employing zosuquidar, daunorubicin, and cytarabine can be employed in treating AML patients other than newly diagnosed AML patients, or for treating other types of leukemia or other cancers that exhibit P-gp expression, as discussed above.

Zosuquidar, daunorubicin, and cytarabine can be formulated as described above for zosuquidar and Mylotarg, and can be included in kits, also as described above.

The zosuquidar, daunorubicin, and/or cytarabine can be to patients suffering from AML prior to confirmation of the P-gp expression or function, or to AML patients other than newly diagnosed AML patients (e.g., relapsed AML patients). However, therapy is preferably administered to newly diagnosed AML patients. The administration route, amount administered, and frequency of administration can vary depending on the age of the patient, status as relapsed or newly diagnosed AML patient, and severity of the condition.

Contemplated amounts of zosuquidar for intravenous administration to treat newly diagnosed AML are from about 400 mg/day or less to about 1,600 mg/day or more, preferably from about 500, 600, or 700 mg/day to about 900, 1000, 1100, 1200, 1300, 1400, or 1500 mg/day, and most preferably 700 mg/day. In the course of a treatment regimen, the zosuquidar is preferably administered on two, three, or four separate days. The dosage is preferably administered in intravenously continuously over the course of about 6 to about 90 hours, more preferably over the course of about 12, 18, 24, 30, 36, or 42 hours to about 54, 60, 66, 72, 78, or 84 hours, most preferably over about 24 hours, 48 hours, or 72 hours, depending upon the treatment regimen. Preferably the zosuquidar is administered on Day 1 of the treatment regimen. In certain embodiments, additional zosuquidar is administered on Day 2, on Days 2 and 3, or on Days 2, 15, and 16. However, in certain embodiments, one, two, or three or more additional doses can be administered on other days of the treatment regimen.

Contemplated amounts of daunorubicin for intravenous administration to treat newly diagnosed AML are from about 10 mg/m²/day or less to about 100 mg/m²/day or more administered at initiation of zosuquidar infusion or up to about 1, 2, 3, 4, 5, or 6 or more hours after initiation of zosuquidar infusion. The dosage is preferably administered intravenously at a rate of about 25 mg/m²/day or less to about 90 mg/m²/day or more, preferably about 30, 35, or 40 mg/m²/day or less to about 50, 55, 60, 65, 70, 75, 80, or 85 mg/m²/day, and most preferably about 45 mg/m²/day continuously over the course of about 2 or 2.5 days to about 3.5 or 4 days, preferably about 3 days.

Contemplated amounts of cytarabine for intravenous administration to treat newly diagnosed AML patients are from about 10 mg/day or less to about 3,000 mg/day or more administered at initiation of zosuquidar infusion or after initiation of zosuquidar infusion. The dosage is preferably administered intravenously at a rate of about 50 mg/m²/day or less to about 200 mg/m²/day or more, preferably 60, 70, 80, or 90 mg/m²/day or less to about 110, 120, 130, 140, 150, 160, 170, 180, or 190 mg/m²/day, and most preferably about 100 mg/m²/day continuously over the course of about 1, 2, 3, 4, 5, or 6 days up to about 8, 9, or 10 days or more, preferably over about 7 days.

A particularly preferred dosing regimen for newly diagnosed AML includes continuous intravenous administration of 550 mg of zosuquidar over 6 hours (3 days), continuous intravenous administration of cytarabine at a rate of 100 mg/m²/day (7 days), and intravenous administration of daunorubicin at a dose of 45 mg/m²/day (3 days), wherein infusion of daunorubicin is started 1 hour after initiation of zosuquidar infusion. Another particularly preferred dosing regimen includes continuous intravenous administration (preferably about 1 to 24 hours in duration, more preferably about 6 to 24 hours in duration, most preferably about 24 hours in duration) of 500 to 700 mg/day of zosuquidar (3 days), continuous intravenous administration of cytarabine at a rate of 100 mg/m²/day (7 days), and intravenous administration of daunorubicin at a dose of 45 mg/m²/day (3 days), wherein infusion of daunorubicin is started 1 to 4 hours after initiation of zosuquidar infusion. While in the above described embodiments infusion of daunorubicin is started after a specified time period has lapsed after initiation of zosuquidar infusion, in other embodiments other start times can be preferred, e.g., immediately after or during initiation of zosuquidar infusion up to about 1, 2, 3, 4, 5, 6, 7, 8, 9, 10, 11, 12, or more hours after initiation of zosuquidar infusion.

While the above methods of the preferred embodiments have been discussed primarily in connection with the treatment of AML, the methods are also particularly efficacious when P-gp substrates are administered as chemotherapeutic agents in the treatment of other malignancies exhibiting some degree of P-gp expression. For example, such malignancies can include lymphomas, bladder cancer, pancreatic cancer, ovarian cancer, liver cancer, myeloma, lymphocytic leukemia, sarcoma, metastatic breast cancer, and most solid tumors. Chemotherapeutic agents that are P-gp substrates include, but are not limited to, anthracyclines (e.g., doxorubicin, daunorubicin, epirubicin, idarubicin, mitoxantrone), vincas (e.g., vincristine, vinblastine, vinorelbine, vindesine), Topoisomerase-I1 inhibitors (e.g., etoposide, teniposide), taxanes (e.g., paclitaxel, docetaxel), and others (e.g., Gleevec, Mylotarg, dactinomycin, mithramycin).

Efflux Assay

A commonly used method for the detection of MDR1 functional activity is referred to as the “efflux assay.” In this assay the tumor cells are incubated with a fluorescent dye such as DiOC2 or Rhodamine 123 to allow for accumulation of the dye. The cells are then washed, a fraction of the cells are measured for dye content immediately or are placed on ice to obtain a baseline dye uptake control, and the remaining cells are recultured in the presence and absence of an MDR1 inhibitor, such as zosuquidar. Since zosuquidar inhibits MDR1, the dye will be retained by such treated cells, and with cells cultured in the absence of zosuquidar the dye will be eliminated (effluxed). Retention and elimination of the dye is quantified by flow cytometry. There is a second way to detect retention or elimination of the dye and it is called the “accumulation assay.” The cells in this assay are incubated with dye in the presence and absence of an MDR1 inhibitor such as zosuquidar. At the end of the incubation period, the cells are washed and assessed for retention of dye by flow cytometry. The standard method for detection of MDR1 activity is by the efflux assay. As shown herein, the accumulation assay is superior to the standard efflux assay for the assessment of MDR1 status in cancer patients. However, the efflux assay can yield substantially improved signal to noise if the cells are allowed to accumulate dye in the presence of an MDR1 inhibitor such as zosuquidar.

Identification of the Distinct Advantage of the Accumulation Assay Over the Standard Efflux Assays Using K562/R7 MDR1-positive and K562 MDR1-negative Cell Lines

K562/R7 cells express MDR1 as a consequence of selection in the presence of doxorubicin. The parental K562 cell line does not express appreciable MDR1. DiOC2 was the dye selected as the surrogate fluorescent marker for MDR1 substrate chemotherapeutic agents. The accumulation assay involved cells cultured at 5×10⁵ cells/ml, 0.5 ml/culture volume, 60 ng/ml DiOC2, and incubation at 37° C for at least 30 and not longer than 90 minutes. The efflux assay involved culturing cells with 60 ng/ml DiOC2, washing the cells and reculturing as described for the accumulation assay. The cells are then analyzed for fluorescence by flow cytometry.

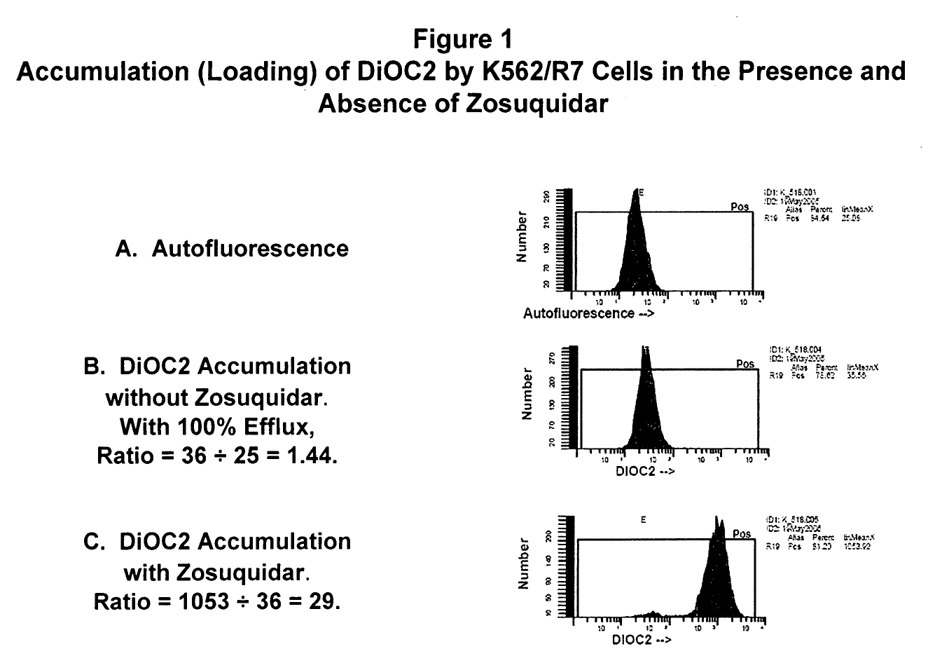

FIG. 1 presents typical histograms of K562/R7. The upper panel shows autofluorescence, i.e., cells cultured in the absence of DiOC2. K562/R7 cells exhibit such strong MDR1 activity and, as shown in the middle panel, accumulated minimal dye in the absence of zosuquidar. However, the cells could be forced to accumulate dye if cultured in the presence of zosuquidar (bottom panel). This illustrates the differences between the typical efflux and accumulation assays. In the typical efflux assay, the amount of dye ultimately loaded into the cells reflects the equilibrium between accumulation and efflux processes. In the accumulation assay with zosuquidar present, the amount of dye ultimately loaded into the cells reflects only influx since efflux is inhibited.

The data are typically expressed as ratios of mean fluorescence intensity (MFI) of zosuquidar-treated cultures divided by the MFI of untreated cultures. In the accumulation assay, the inhibitory effects of zosuquidar on K562/R7 MDR1 function were clearly evident as indicated by the inhibition ratio of 15.6. In the efflux assay, assuming complete elimination of the dye to autofluorescence levels, a ratio of 1.55 would be observed, which is considered dim or negative MDR1 activity with the standard efflux assay. Thus, as will be addressed subsequently, it is quite possible that leukemia cells with very high MDR1 activity could similarly be misclassified with the standard efflux assay.

The Distinct Advantage of the Accumulation Assay Over the Standard Efflux Assay can be Observed with Leukemia Cells

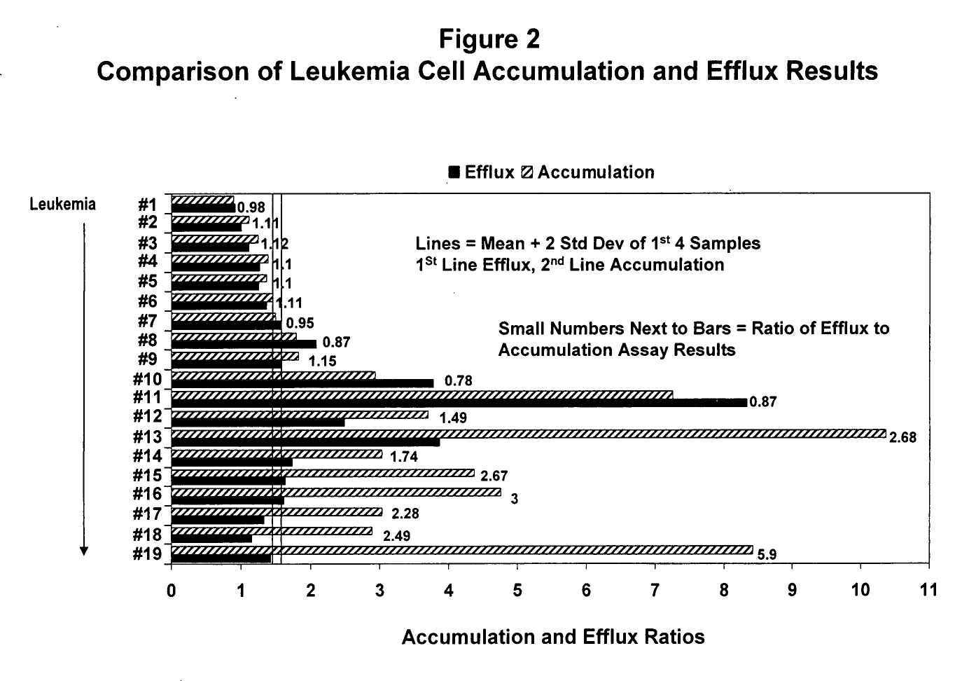

A comparison was conducted between the accumulation and efflux assays with leukemia cells. FIG. 2 presents a graphic depiction of the data. Negative or “dim” results are indicated by samples with ratios to the left of the vertical line at a ratio of 1.55. A number of samples exhibited comparable ratios in the two assays. However, some samples exhibited what would be considered dim or negative MDR1 functional activity in the efflux assay, but rather substantial activity in the accumulation assay. While not wishing to be bound by theory, a possible explanation for this observation could be that in the accumulation assay some highly active MDR1-positive cells, like K562/R7 cells (referring to FIG. 1), can be forced to accumulate dye provided that zosuquidar is present. In the efflux assay, where zosuquidar is not present during dye loading, these highly active cells would not accumulate enough dye to be identified as effluxing during the secondary efflux stage of that assay.

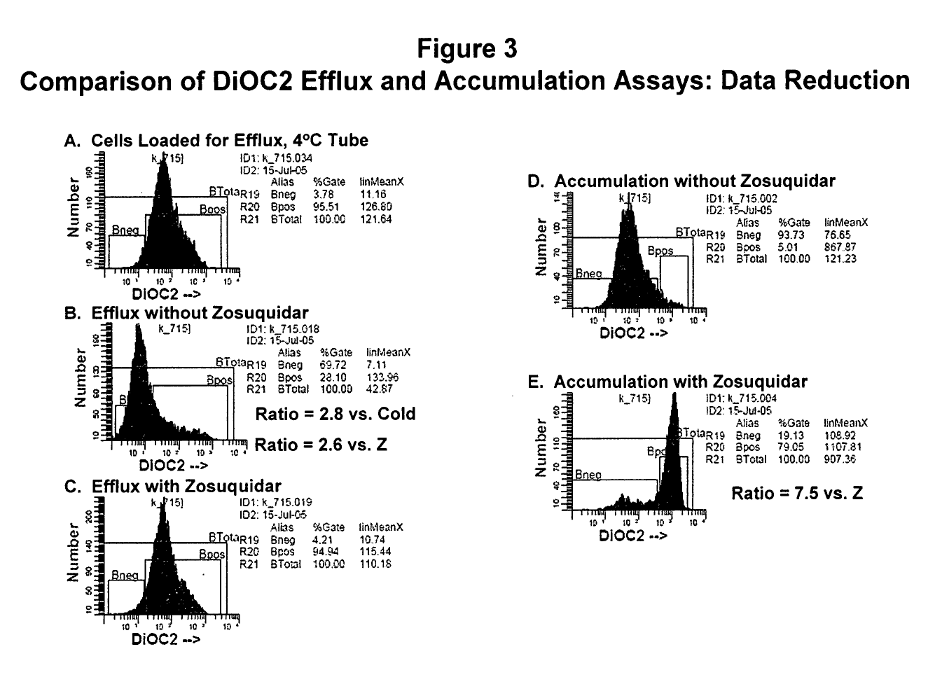

FIG. 3 presents an example of these phenomena in a leukemia cell sample. The cells were loaded for the efflux assay and either placed on ice (upper left panel, baseline control) or allowed to efflux in the absence (middle left panel) or presence of zosuquidar (lower left panel). The efflux ratio was 2.6. The maximum amount of dye in the loaded cells yielded a MFI=121. However, as shown in the lower right panel, loading in the presence of zosuquidar (accumulation) yielded a MFI=907 and a ratio=7.5. The data can be interpreted as demonstrating, as with K562/R7 cells, that leukemia cells with high MDR1 activity only accumulate substantial amounts of dye when cultured in the presence of an MDR1 inhibitor.

The Use of Zosuquidar during Dye Loading Can Yield a Highly Sensitive Efflux Assay

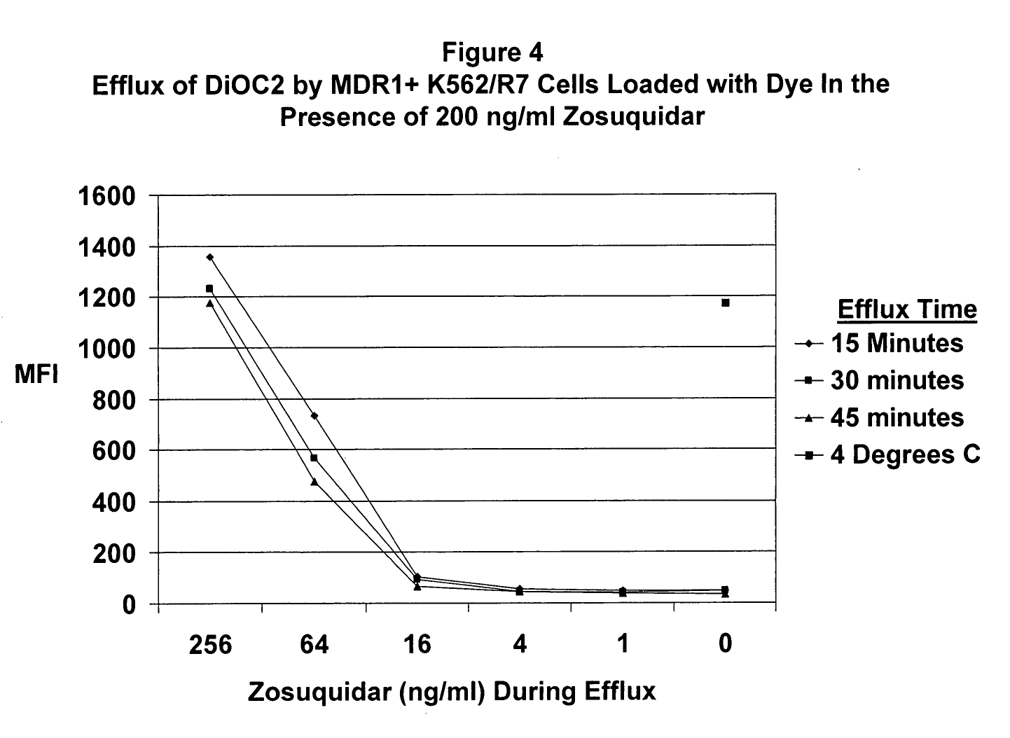

FIG. 4 presents the efflux characteristics of K562/R7 cells which had been loaded with 40 ng/ml DiOC2 in the presence of 200 ng/ml zosuquidar for 60 minutes. The loaded and washed cells were cultured at 37° C. in 0.5 ml lacking or containing the indicated concentrations of drug for the indicated amount of time. Control cells were placed on ice immediately after loading and, as indicated by the symbol to the far right at MFI=1172, the cells had been loaded with dye. Incubation (efflux) in the presence of zosuquidar inhibited dye release in a concentration-dependent manner which was basically independent of incubation period. Significantly, in contrast to the low efflux ratio (1.55) illustrated in FIG. 1 using the standard efflux methods, loading cells in the presence of zosuquidar yielded an efflux assay with a high efflux ratio=26.8.

Discussion of Results

As shown herein, the standard efflux method suffers from a lack of sensitivity when leukemia cells with very high MDR1 activity are tested. The amount of dye present in cells to be tested in the standard efflux assay reflects the equilibrium between accumulation and efflux during the loading period. The poor sensitivity of the standard efflux assay results from the high efflux rate of some cancer samples yielding poorly loaded cells for the test. In other words, the presence of leukemia cells with the highest MDR1 activity would be missed with the standard efflux assay because they were poorly loaded with dye. The present results show that the accumulation assay, which reflects accumulation which is independent of efflux (inhibited by zosuquidar), frequently yielded a much more sensitive assay for the assessment of leukemia cells with high MDR1 activity. Furthermore, by loading such cells in the presence of an MDR1 inhibitor such as zosuquidar, an enhanced efflux assay was achieved (FIG. 1 vs. FIG. 4). These novel observations indicate that the use of the accumulation assay, or the efflux assay with cells loaded with dye in the presence of zosuquidar, are far superior methods over the standard efflux assay, for the assessment of MDR1 status in patients with cancer.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. A method of treating a condition in a patient by administering a therapeutic agent that is a substrate for P-glycoprotein efflux, the method comprising the steps of:
determining P-glycoprotein expression or P-glycoprotein function; and
administering the therapeutic agent in combination with a P-glycoprotein efflux pump inhibitor when P-gp expression is positive.

2. The method of claim 1, wherein P-glycoprotein expression is determined by an antibody assay, and wherein positive P-glycoprotein expression is observed in at least about 10% of the cells tested in the antibody assay.

3. The method of claim 2, wherein P-glycoprotein expression is determined by an antibody assay, and wherein positive P-glycoprotein expression is observed in from about 10% to about 25% of the cells tested in the antibody assay.

4. The method of claim 1, wherein P-glycoprotein function is determined by a P-glycoprotein dye accumulation assay, and wherein a ratio of dye taken up by cells in the presence of the P-glycoprotein efflux pump inhibitor to dye taken up by cells cultured in the absence of the P-glycoprotein efflux pump inhibitor is at least about 1:1.2.

5. The method of claim 4, wherein P-glycoprotein function is determined by a P-glycoprotein dye accumulation assay, and wherein a ratio of dye taken up by cells in the presence of the P-glycoprotein efflux pump inhibitor to dye taken up by cells cultured in the absence of the P-glycoprotein efflux pump inhibitor is from about 1:1.2 to about 1:1.5.

6. The method of claim 1, wherein P-glycoprotein function is determined by a P-glycoprotein dye efflux assay, and wherein an amount of dye eliminated from the cells in the presence of the P-glycoprotein efflux pump inhibitor divided by an amount of dye eliminated in the absence of P-glycoprotein efflux pump inhibitor is at least about 1:1.2.

7. The method of claim 6, wherein P-glycoprotein function is determined by a P-glycoprotein dye efflux assay, and wherein an amount of dye eliminated from the cells in the presence of the P-glycoprotein efflux pump inhibitor divided by an amount of dye eliminated in the absence of P-glycoprotein efflux pump inhibitor is from about 1:1.2 to about 1:1.5.

8. The method of claim 1, wherein P-glycoprotein function is determined by a P-glycoprotein dye efflux assay, and wherein an amount of dye eliminated from the cells in an absence of the P-glycoprotein efflux pump inhibitor is at least about 30% higher than that contained in baseline control cells, and wherein cells incubated in a presence of the P-glycoprotein efflux pump inhibitor have dye levels at least about 30% higher than cells cultured in an absence of the P-glycoprotein efflux pump inhibitor.

9. The method of claim 1, wherein the P-glycoprotein efflux pump inhibitor is selected from the group consisting of zosuquidar, Tariquidar, and Tesmilifene.

10. The method of claim 1, wherein the therapeutic agent comprises an immunosuppressant.

11. The method of claim 10, wherein the immunosuppressant is selected from the group consisting of cyclosporine, cyclosporine A, and tacrolimus.

12. The method of claim 1, wherein the therapeutic agent comprises a steroid.

13. The method of claim 12, wherein the steroid is selected from the group consisting of dexamethasone, hydrocortisone, corticosterone, triamcinolone, aldosterone, and methylprednisolone.

14. The method of claim 1, wherein the therapeutic agent comprises an antiepileptic.

15. The method of claim 14, wherein the antiepileptic comprises phenytoin.

16. The method of claim 1, wherein the therapeutic agent comprises an antidepressant.

17. The method of claim 16, wherein the antidepressant is selected from the group consisting of citalopram, thioperidone, trazodone, trimipramine, amitriptyline, and phenothiazines.

18. The method of claim 1, wherein the therapeutic agent comprises an antipsychotic.

19. The method of claim 18, wherein the antipsychotic is selected from the group consisting of fluphenazine, haloperidol, thioridazine, and trimipramine.

20. The method of claim 1, wherein the therapeutic agent comprises a protease inhibitor.

21. The method of claim 20, wherein the protease inhibitor is selected from the group consisting of amprenavir, indinavir, lopinavir, nelfinavir, ritonavir, and saquinavir.

22. The method of claim 1, wherein the therapeutic agent comprises a calcium blocker.

23. The method of claim 22, wherein the calcium blocker is selected from the group consisting of bepridil, diltiazem, flunarizine, lomerizine, secoverine, tamolarizine, verapamil, nicardipine, prenylamine, and fendiline.

24. The method of claim 1, wherein the therapeutic agent comprises a cardiac drug.

25. The method of claim 24, wherein the cardiac drug is selected from the group consisting of digoxin, diltiazem, verapamil, and talinolol.

26. A pharmaceutical kit, the kit comprising:
at least one dose of a P-glycoprotein efflux pump inhibitor selected from the group consisting of zosuquidar, Tariquidar, and Tesmilifene;
directions for conducting a diagnostic for determining a value for P-gp expression associated with a condition; and
directions for administering the P-glycoprotein efflux pump inhibitor and a therapeutic agent that is a substrate for P-glycoprotein efflux to the patient to treat the condition when P-glycoprotein expression or P-glycoprotein function is positive.
